(19) United States
(12) Patent Application Publication
(10) Pub. No.: US 2023/0037786 A1
(43) Pub. Date: Feb. 9, 2023

(54) METHOD AND SYSTEM FOR RECEIVING A DEBT PAYMENT

(71) Applicant: CREDIT CLEAR INTERNATIONAL PTY LTD, Victoria (AU)

(72) Inventor: Trent MCKENDRICK, Victoria (AU)

(21) Appl. No.: 17/936,812

(22) Filed: Sep. 29, 2022

Related U.S. Application Data
(63) Continuation-in-part of application No. 16/076,725, filed on Aug. 9, 2018.

(51) Int. Cl. G06Q 20/10; G06Q 40/02; G06Q 30/02; A61B 5/16; H04L 51/046

(52) U.S. Cl. CPC G06Q 20/102; G06Q 40/025; G06Q 30/0235; A61B 5/165; H04L 51/046

(57) ABSTRACT

A method of determining a likelihood that a debt payment will be made or received, the method comprising sending a message to a debtor mobile computing device comprising a link to a debtor portal; displaying on the debtor mobile computing device an indication of the debt owing; displaying on the debtor mobile computing device a proposed payment plan for settlement of the debt; and receiving an indication that the debtor accepts the proposed payment plan is disclosed. The method may use Next Best Action (NBA) modelling. The message sent may be a SMS message and it may include an indication of the debt owing. The method may further comprise offering a discount to the debtor if the discounted amount is paid that day. The offer of a discount may initiate an automated negotiation process with the debtor.

### RELATED APPLICATIONS

This application is a continuation in part of U.S. patent application Ser. No. 16/076,725 filed Aug. 9, 2018, which is a national phase of International Application No. PCT/AU2016/000035 filed Feb. 10, 2016 and published in English under International Publication No. WO2017/136865 on Aug. 17, 2017, each of which is incorporated herein by reference.

### FIELD OF THE INVENTION

The present invention relates to a method and system for determining the likelihood of a debt payment being made or received. More particularly, the invention relates to a method and system for determining the likelihood of a debt payment being made or received which uses a Next Best Action (NBA) model.

### BACKGROUND TO THE INVENTION

The majority of debt and arrears collections by various providers, including bank and non-bank lenders, credit card providers, services and utilities companies, telecommunications and any business or person providing business or consumer invoicing or business invoicing accounts, are currently undertaken by outsourcing to third party collectors, with lenders forfeiting around 40% of the amount owing to collection agencies in order to collect the debt. In 2014 alone, debt collection agencies in Australia recovered approximately 2.2 bn in debts, costing lenders or debtors 880 m in revenues.

By contrast, a digital approach allows vastly more efficient communications. The market for such products continues to grow, as volumes of small and medium loans to borrowers have increased over the past two decades. As such, several providers have expressed a significant interest in a digital service to improve efficiency in communication with, and collection of debts from, borrowers, consumers and businesses.

The debt collection process is not often viewed as a driver of customer satisfaction. The reality of talking to debtors in difficult situations, coupled with tight regulations which includes state and federal regulations, coupled with internal processes, leads to numerous pitfalls that requires complicated navigation. For a variety of reasons around personal interactions concerning debt and emotion, often debtors are not willing to talk to a personal representative from the debtee or their agent. For this reason, there is a need for alternative business models and solutions which reflect the goals of informing consumers or businesses and reducing the incidence of misleading, unconscionable, and coercive tactics by collections departments or external agencies.

Accordingly, improved methods for paying debts and receiving these payments are required.

### SUMMARY OF THE INVENTION

The present invention is broadly directed to a method and system for determining the likelihood that a debt payment will be made or received.

In a broad form, the invention relates to a method and system for determining the likelihood that a debt payment will be made or received. In some forms, the present invention is of particular advantage by utilizing a Next Best Action (NBA) model.

In a first aspect, the present invention provides a method of receiving a debt payment comprising:

sending a message to a debtor mobile computing device comprising a link to a debtor portal;

displaying on the debtor mobile computing device an indication of the debt owing;

displaying on the debtor mobile computing device a proposed payment plan for settlement of the debt; and

receiving an indication that the debtor accepts the proposed payment plan.

The message sent may comprise an electronic message such as, an SMS message, an instant message or a direct message. The message may be sent and received on any suitable platform such as, WhatsApp; Line; Wechat; Facebook messenger; Instagram messaging or TikTok.

The message may further comprise an indication of the debt owing.

The method may further comprise the step of offering a discount to the debtor if the discounted amount is paid that day. The offer of a discount may initiate an automated negotiation process with the debtor. The automated negotiation process may comprise pre-set discount parameters. The present discount parameters may be modified to provide one or more additional payment incentive. The modification may comprise a limited time offer.

The method may further comprise a step of learning from debtor behaviour. The learning may comprise one or more of machine learning, behavioural analytics and a humanist approach.

The method may further comprise collecting behavioural data and using A/B testing to determine the most effective message to send to a debtor.

The method may further comprise storing data or metadata on debtor activity.

The method may further comprise one or more algorithm which collect data on user behaviour, and a suggestive analytics framework which takes this user data and determines the most effective communication strategy for debt recovery.

In a second aspect, the present invention provides a server based system for receiving a debt payment comprising:

a network connected messaging processor for sending a message to a debtor mobile computing device, the sent message comprising a link to a debtor portal;

a portal for displaying on the debtor mobile computing device an indication of the debt owing and for displaying on the debtor mobile computing device a proposed payment plan for settlement of the debt; and

a server adapted to interface with the debtor mobile computing device for receiving an indication that the debtor accepts the proposed payment plan.

The message sent by the processor may comprise an electronic message such as, a SMS message, an instant message or a direct message. The message may be sent and received on any suitable platform such as, WhatsApp; Line; Wechat; Facebook messenger; Instagram messaging or TikTok.

The message sent by the processor may further comprise an indication of the debt owing.

The portal may further offer a discount to the debtor if the discounted amount is paid that day. The offer of a discount may initiate an automated negotiation process with the debtor. The automated negotiation process may comprise pre-set discount parameters. The present discount parameters may be modified to provide one or more additional payment incentive. The modification may comprise a limited time offer.

The portal may further learn from debtor behaviour. The learning may comprise one or more of machine learning, behavioural analytics and a humanist approach.

The portal may further collect behavioural data and use A/B testing to determine the most effective message to send to a debtor.

The system may further comprise one or more database to store data or metadata on debtor activity.

The portal may further utilise one or more algorithm which collect data on user behaviour, and a suggestive analytics framework which takes this user data and determines the most effective communication strategy for debt recovery.

In a third aspect, the present invention provides a computer program product for receiving a debt payment, the computer program product comprising a computer usable medium and computer readable program code embodied on said computer usable medium, the computer readable code comprising: computer readable program code devices (i) configured to cause the computer to send a message to a debtor mobile computing device, the sent message comprising a link to a debtor portal;

computer readable program code devices (ii) configured to cause the computer to display on the debtor mobile computing device an indication of the debt owing and to display on the debtor mobile computing device a proposed payment plan for settlement of the debt;

and computer readable program code devices (iii) configured to cause the computer to receive an indication that the debtor accepts the proposed payment plan.

The message sent may comprise an electronic message such as, a SMS message, an instant message or a direct message. The message may be sent and received on any suitable platform such as, WhatsApp; Line; Wechat; Facebook messenger; Instagram messaging or TikTok.

The message sent may further comprise an indication of the debt owing.

The display may further comprise an offer of a discount to the debtor if the discounted amount is paid that day. The offer of a discount may initiate an automated negotiation process with the debtor. The automated negotiation process may comprise pre-set discount parameters. The present discount parameters may be modified to provide one or more additional payment incentive. The modification may comprise a limited time offer.

The computer program product may further comprise computer readable program code devices (iv) configured to cause the computer to learn from debtor behaviour. The learning may comprise one or more of machine learning, behavioural analytics and a humanist approach.

The computer program product may further comprise computer readable program code devices (v) configured to cause the computer to collect behavioural data and use A/B testing to determine the most effective message to send to a debtor.

The computer program product may further comprise computer readable program code devices (vi) configured to cause the computer to interface with one or more network connected database to store data or metadata on debtor activity.

The computer program product may further comprise computer readable program code devices (vii) configured to cause the computer to utilise one or more algorithm which collect data on user behaviour, and a suggestive analytics framework which takes this user data and determines the most effective communication strategy for debt recovery.

Any of the above aspects may be applied to any type of debtor. The type of debtor may comprise a non-bank debtor, a utility debtor, a telecom debtor, a personal loan debtor, a business loan debtor and a professional services debtor.

According to any one of the above aspects, a Next Best Action (NBA) model is comprised. The NBA model may be dynamically adjusted. The dynamic adjustment may be based on one or more of specified data and events post loading. The NBA model may be self correcting. The NBA model may predict one or more new behaviour based on the collected behavioural data. For example, message and communication may be tailored towards the specific debtor and responses to events and/or interaction.

The NBA model may predict one or more debtor behaviour and provide guidance on best next course of action.

The NBA model may comprise a temporal component such as, selection of a time of day the message is sent.

The NBA model may be generalised for multiple implementations.

The NBA model may adjust one or more prediction based on the specified data such as, provided client information. The provided client information may comprise one or more of client type; debt type; one or more client attribute and debt delinquency.

The NBA model may adjust one or more prediction based on provided debtor information such as, known demographic data; regional demographic data; and debt specifics.

The NBA model may adjust one or more prediction based on one or more outcomes of past events such as, digital delivery success metrics; and dialler calls success metrics.

The NBA model may tailor messaging tone and/or contents to debtor and the debtor's history.

The NBA model may adjust one or more prediction based on the events post loading. The events post loading may comprise one or more of debtor engagement such as, engagement with prior messages (click/open rates); payment portal viewing; inbounds calls received; arrangements set; payments made; credit clear portal interactions.

The NBA model may support testing of new and/or altered actions such as, activity outside conventional work hours, which may include Saturday activity and/or variable messaging platforms such as, WhatsApp; LINE or WECHAT messaging.

The NBA model may comprise the ability to use prior known information such as, previous referrals of same debtor, for predictions such as, past sequence of events leading to payment.

The NBA model may link payments to actions and measure how much that payment can be attributed to each action.

The NBA model may comprise a Recurrent Neural Network (RNN). The RNN may comprise an internal memory to remember an input. The internal memory may be used to integrate the temporal component into the modelling and/or to utilise one or more fixed data points.

According to any one of the above aspects actions may be limited to ‘SMS’, ‘Email’ and ‘Outbound Dialler Calls’.

In another embodiment of any one of the above aspects, TensorFlow may be utilised to build the NBA model and/or LTSM layers was used.

In yet another embodiment of any one of the above aspects the NBA model may make a prediction from 0 to 1 of conversion based on a specific action being taken on evaluation. The NBA model may determine a probability distribution by providing multiple actions across all variations.

An action may be selected based on the determined distribution. The specific action may comprise a type of message such as, contact by SMS or contact by email. The action with the highest probability may be selected.

In still another embodiment of any one of the above aspects the NBA model may use a binary classification problem such as, either the debtor converts or doesn't. Each variation of an action may be provided individually.

The binary classification model may comprise of three distinct models; one for each of communication type, send time and message contents. The sequence and customer data to each model may be provided separately and then an evaluation for each possible option of each model conducted. The evaluation may provide a probability distribution from each model which may be combined together with a calibration layer to provide a probability of each possible action.

In one embodiment of any one of the above aspects the NBA model may measure a likelihood of an account balance being fully cleared by a fixed date from the account being loaded if a specific action is taken.

In another embodiment, the NBA model may determine a chance of conversion of an account, payment or arrangement set up, within 7 days if action was taken today.

In one embodiment, Shapley Values are used in order to attribute how actions led to conversion.

In another embodiment, the NBA model may be split into sub models such as, a preferred contact step; a message contents step; and a send time optimisation step.

Where the terms “comprise”, comprises”, “comprising”, “include”, “includes”, “included” or “including” are used in this specification, they are to be interpreted as specifying the presence of the stated features, integers, steps, or components referred to, but not to preclude the presence or addition of one or more other feature, integer, step, component or group thereof.

Further, any prior art reference or statement provided in the specification is not to be taken as an admission that such art constitutes, or is to be understood as constituting, part of the common general knowledge.

### BRIEF DESCRIPTION OF THE FIGURES

In order that the present invention may be readily understood and put into practical effect, reference will now be made to the accompanying illustrations, wherein like reference numerals refer to like features and wherein:

FIG. 1A shows a flowchart showing the steps according to one embodiment of the invention.

FIG. 1B shows a smartphone displaying one embodiment of a message sent according to the invention.

FIG. 1C shows the smartphone displaying one embodiment of the debtor portal according to the invention.

FIGS. 2A and 2B: shows a graphical representation of one embodiment of a personal mobile computing device suitable for use with the invention.

FIG. 3 shows one example of an operator dashboard according to the invention.

FIG. 4 shows an example of block diagram.

FIG. 5A shows an example graph of conversion rate since days past referral.

FIG. 5B shows an example graph of collection rate since days past referral.

FIG. 6 shows an example graph of modelled uplift during experiment run.

### DETAILED DESCRIPTION OF THE INVENTION

The present invention relates to a method and system for receiving a debt payment. The method and system of the present invention find particular application in the pre-default stage of collections, which is between an overdue account being manually handled by providers and the use of any external collections agency.

Just one significant advantage of the present claimed invention is that it may be applied to any type of debtor. Just some examples of the type of debtor they may benefit from the present claimed invention are a non-bank debtor, a utility debtor, a telecom debtor, a personal loan debtor, a business loan debtor and a professional services debtor.

In one embodiment, the present invention is partly predicated on the present inventor's diligent study which has resulted in the discovery that application of a game interface provides a positive environment for a debtee which encourages them to pay their debts.

As will be evident from the below, the present invention provides the combination of a simple user experience and innovative communication algorithms which is of particular advantage because it provides a pleasant interface which allows debts to be settled in a non-confronting way.

The presently claimed invention takes advantage of the high success rate of digital communications by utilising SMS and email contact and providing a debtor-facing web portal that gives debtors clear, simple information about what is owed and what options are available for repayment. This portal may make clear the debtor's legal rights and obligations, and provides the tools and information needed to manage and clear their overdue accounts.

As used herein “A/B testing” or “split testing” comprises comparing two versions of a web page such as, the debtor portal to see which one performs better. The two versions of the web page or debtor portal are shown to similar visitors or debtors. The one that gives a better performance such as, a better conversion rate, is implemented.

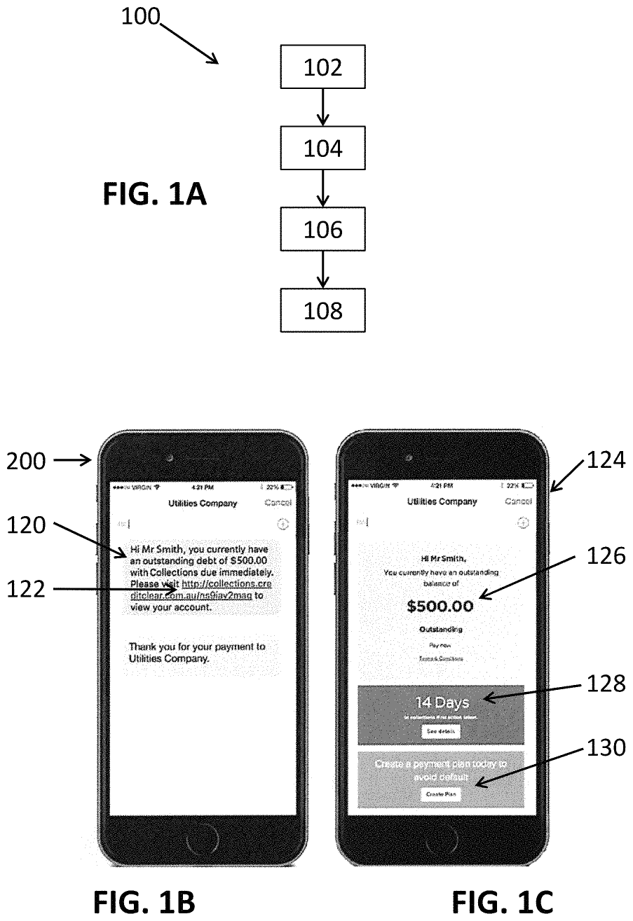

As shown in FIGS. 1A, 1B and 1C, in one embodiment the method 100 of the invention comprises the step 102 of sending a message 120 to a debtor mobile computing device 200. In the embodiment shown, message 120 comprises a SMS, which is of surprising advantage in payment compliance due to the efficacy of SMS communication.

As shown in FIG. 1B, message 120 may comprise a link 122 to a borrower-facing debtor portal 124 that is displayed on debtor mobile computing device 200. The borrow-facing debtor portal 124 may operate with minimal input required from lenders or debtees. In the embodiment shown, the link 122 comprises a hyperlink.

By pressing link 122, borrower-facing debtor portal 124 comprising an indication of the debt owing 126, the deadline for payment 128 and a button 130 to display a proposed payment plan for settlement of the debt is displayed on debtor mobile computer device 200.

A debtor accepts the proposed payment plan by pressing an accept button 132 (not shown) displayed on debtor mobile computing device 200 and this acceptance is sent to and received server 291.

The method 100 may further comprise the step of offering a discount to the debtor if the discounted amount is paid that day. The offer of a discount may initiate an automated negotiation process with the debtor. The automated negotiation process may comprise pre-set discount parameters. The present discount parameters may be modified to provide one or more additional payment incentive. The modification may comprise a limited time offer.

In one embodiment, the method comprises a step of learning from debtor behaviour. The learning may comprise one or more of machine learning, behavioural analytics and a humanist approach.

The method may further comprise collecting behavioural data and using A/B testing to determine the most effective message to send to a debtor

The method may further comprise storing data or metadata on debtor activity.

The method may further comprise one or more algorithm which collect data on user behaviour, and a suggestive analytics framework which takes this user data and determines the most effective communication strategy for debt.

The presently claimed invention may be of particular advantage by allowing borrower behaviour to be measured to identify behavioural trends, leading to data and insights on the most effective means of communicating with borrowers based on their behaviour and demographics.

The presently claimed invention is of surprising advantage because it makes use of a combination of learning algorithms and suggestive analytics. The method and system of the invention may be configured with pre-set ‘rules of engagement’, the method or system may automatically determine the most effective method for reaching the debtee and contacts them for example, by email or SMS, with a link to the debtor portal. The borrower portal of the presently claimed invention is of particular advantage because it allows borrowers in straightforward cases to clear their debt without needing to speak with a collector, thereby freeing up lender resources to focus on more difficult cases. This is of surprising advantage because of its efficacy in attracting payment.

The presently claimed invention may also be an intelligent system, giving debtors the capability to collect and analyse data on their collections processes in order to take advantage of the opportunities for increasing efficiency that digital solutions provide. The presently claimed invention's debtor portal collects behavioural data and allows A/B testing to determine the most effective ways to contact borrowers. By basing decision strategy on machine learning and borrower behaviour, the presently claimed invention may assist debtees find the fewest number of contact points with the highest recovery rate, minimising cost for the lender and nuisance to the borrower.

Advantageously, the present invention also provides a Next Best Action (NBA) Model. Current collection strategies follow a linear model where collection actions are planned and mapped out by days since a fixed point in time. There is some branching which occurs but this is limited. Some decisioning is used with a high/medium/low risk classification or alternative contacts used in the case of missing contact points (mobile instead of email etc).

Advantageously, in order to make improvements the inventors have realised that testing must be done on different events and combinations for each client. This is time consuming and costly.

Ideally, when a new implementation is made a best approach model that is dynamically adjusted based on specified data and events post loading would be used. This model would be self correcting and learn and predict new behaviour based on the latest data. For example, message and communication is tailored towards the specific debtor and responds to events/interaction.

The NBA model looks to predict debtor behaviour and provide guidance on best next course of action. This is outlined further below.

The NBA has a set of model features. These comprise a temporal component. It is known from prior analytics that debtor behaviour is influenced by timing/frequency of events. This includes the time of day the message is sent.

Another feature is that the NBA model is able to be generalised for multiple implementations. Advantageously, this avoids servicing a model for each implementation.

Another feature is that ability to adjust predictions based on the specified data such as, provided client information which may comprise one or more of client type; debt type; one or more client attriand debt delinquency.

Yet another feature is the ability to adjust predictions based on provided debtor information such as known demographic data; regional demographic data; and debt specifics.

Still another feature is the ability to adjust predictions based on outcomes of past events such as digital delivery success metrics; and dialler calls success metrics.

Another feature is the ability to tailor messaging tone and/or contents to debtor and the debtor's history.

Still another feature is the ability to adjust predictions based on the events post loading such as one or more of: debtor engagement such as, engagement with prior messages (click/open rates); payment portal viewing; inbounds calls received; arrangements set; payments made; credit clear portal interactions.

Yet another feature is the support of testing of new and altered actions such as, activity outside conventional work hours, which may include Saturday activity and/or variable messaging platforms such as, WhatsApp; LINE or WECHAT messaging.

Another feature is the ability to use prior known information such as, previous referrals of same debtor, for predictions such as, past sequence of events leading to payment.

Advantageously, the invention provides the ability to link payments to actions and measure how much that payment can be attributed to each action.

In one embodiment, a Recurrent Neural Network (RNN) is used. RNNS are the state of the art algorithm for sequential data. RNNs remembers its input, due to an internal memory, which makes it suited to machine learning problems that involve sequential data.

Advantageously, this approach allows the integration of the temporal component of the strategy into the modelling, as well as the utilisation of all usual fixed data points.

In one embodiment actions may be limited to ‘SMS’, ‘Email’ and ‘Outbound Dialler Calls’.

In another embodiment, TensorFlow may be utilised to building a model and Long Short Term Memory (LTSM) layers may be used.

In one particular embodiment the model makes a prediction from 0 to 1 of conversion based on specific action being taken on evaluation. By providing multiple actions across all variations the method of the invention can find the probability distribution and select an action based on this distribution. The specific action may comprise a type of message such as, contact by SMS or contact by email. The action with the highest probability may be selected.

A binary classification problem may be used, either the debtor converts or doesn't. Accordingly, each variation of the action needs to be provided individually rather than a multiclass classification where each action is represented by its own class. Although slower to evaluate, the binary classification approach is simpler to train and allows for better scaling of variations in actions.

The binary classification model may comprise of three distinct models; one for each of communication type, send time and message contents. The sequence and customer data to each model may be provided separately and then an evaluation for each possible option of each model conducted. The evaluation may provide a probability distribution from each model which may be combined together with a calibration layer to provide a probability of each possible action. They may be similar to the outcome vector for the multiclass classification.

In one embodiment the likelihood of an account balance being fully cleared by a fixed date from the account being loaded if a specific action was taken was measured. There were two issues with this approach. The outcome at X number of days wasn't only dependent on the status of the account at present (when the model is making a decision). Further activity post the current state of the model would influence the final outcome and the model learnt this behaviour indirectly even when this data wasn't provided to the model when making predictions. This was difficult to generalise for multiple clients. This fixed date would be client specific and would need to become a data point in the model itself.

In another embodiment, the model determines chance of conversion of an account, payment or arrangement set up, within 7 days if action was taken today. A potential downside of this approach is that as assumption is made regarding an improvement in conversion now leading to an improvement in long term collection and recovery rates. This is something we can measure as part of testing but was not part of the model building itself.

In one embodiment, Shapley Values are used in order to attribute how actions led to conversion.

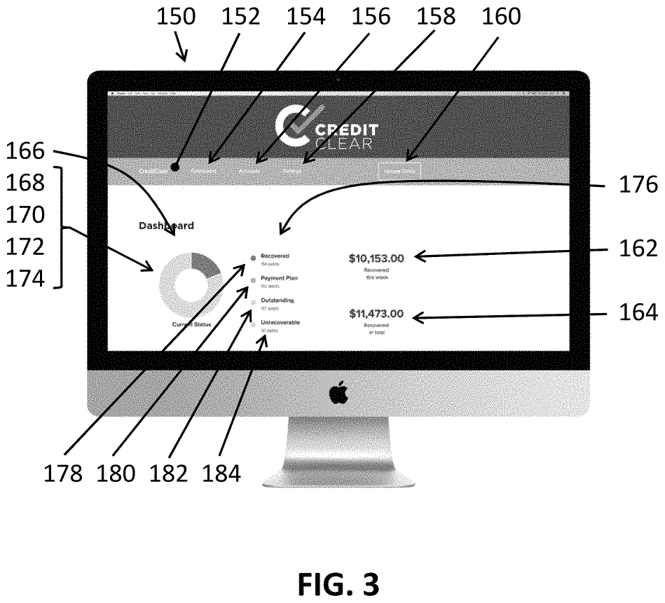

In another embodiment, the NBA model may be split into sub models. One example of this is shown in FIG. 4. For example, the NBA model may be split into a preferred contact step; a message contents step; and a send time optimisation step.

Improving the borrower's experience is a core part of the present invention. At present, a full third of all complaints about debt collection relate to harassment and coercion by collectors. As a compliance-driven and strictly regulated industry, it is unacceptable to have such a large volume of complaints be related to unscrupulous behaviour. To address this, the presently claimed invention complies with the relevant regulatory frameworks, ensuring contact with borrowers is compliant with regulations and applicable Codes. As such, the presently claimed invention prevents unscrupulous collectors from applying undue pressure to borrowers.

This highlights another advantage of the present invention, which is that it provides debtees with access to all the relevant information, and to ensure collections comply with regulations and industry guidance.

The present invention provides a service which simplifies and optimises the payment of outstanding accounts, through a simple to understand borrower portal. Through adjustable and pre-set rulesets, the portal 124 communicates with debtors holding arrears accounts with minimal handling required by the debtee. This scalable technology incorporates powerful analytical tools to provide debtees with insights into arrears accounts in order to further optimise their collections process.

Another advantage of the presently claimed invention is that it allows debtors to be able to quickly assess their obligations and easily address them, as they typically wish to end their involvement as quickly as possible. They typically desire more flexibility in responding as time allows, and to be contacted by automated message.

The present inventor has developed a method and system that gives lenders the tools to simply, efficiently and effectively manage their overdue accounts. With its strong focus on analytics and increased debtor satisfaction and control, the data driven approach of the present invention decreases both default rates and the cost of collections, benefiting both lenders and borrowers.

In one embodiment, the present invention is a cloud-based software-as-a-service solution which provides debtees with information and options to take action through an easy to understand web portal. This may be coupled this with a set of real-time analytics tools for debtees designed to analyse borrower behaviour and draw from existing accounting and loan management systems, in order to identify the most efficient and effective means for communicating with customers.

In another embodiment, the present invention provides algorithmic learning capabilities which help analyse specific data to enhance a debtee's ability to make informed decisions on the type of credit offered to borrowers, and to identify borrower risk profiles.

In a particular embodiment, the present invention is used in the pre-default area of collections, with the core focus of activity being early stage reminders to prompt payments through a cloud-based portal.

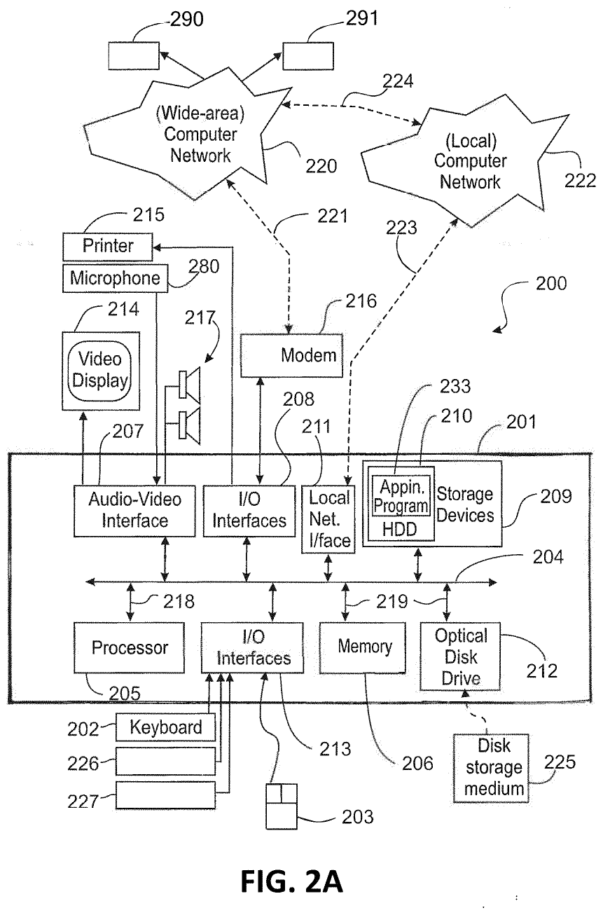

One embodiment of a debtor mobile computing device 200 suitable for use in the present invention is shown in FIGS. 2A and 2B. In the embodiment shown debtor mobile computing device 200 comprises a computer module 201 comprising input devices such as a keyboard 202, a mouse pointer device 203, a scanner 226, a camera 281, an external hard drive 227, a touchscreen video display 214 and a microphone 280; and output devices including a printer 215, the touchscreen video display device 214 and loudspeakers 217. In some embodiments video display 214 may comprise a touchscreen.

A Modulator-Demodulator (Modem) transceiver device 216 may be used by the computer module 201 for communicating to and from a communications network 220 via a connection 221. The network 220 may be a wide-area network (WAN), such as the Internet, a cellular telecommunications network, or a private WAN. Through the network 220, computer module 201 may be connected to; and may have system interaction 293 with; other similar personal mobile computing devices 290 or server computers 292. Where the connection 221 is a telephone line, the modem 216 may be a traditional “dial-up” modem. Alternatively, where the connection 221 is a high capacity (e.g.: cable) connection, the modem 216 may be a broadband modem. A wireless modem may also be used for wireless connection to network 220.

The computer module 201 typically includes at least one processor 205, and a memory 206 for example formed from semiconductor random access memory (RAM) and semiconductor read only memory (ROM). The module 201 also includes a number of input/output (I/O) interfaces including: an audio-video interface 207 that couples to the touchscreen video display 214, loudspeakers 217 and microphone 280; an I/O interface 213 for the keyboard 202, mouse 203, scanner 226 and external hard drive 227; and an interface 208 for the external modem 216 and printer 215. In some implementations, modem 216 may be incorporated within the computer module 201, for example within the interface 208. The computer module 201 also has a local network interface 211 which, via a connection 223, permits coupling of the debtor mobile computing device 200 to a local computer network 222, known as a Local Area Network (LAN).

As also illustrated, the local network 222 may also couple to the wide network 220 via a connection 224, which would typically include a so-called “firewall” device or device of similar functionality. The interface 211 may be formed by an Ethernet circuit card, a Bluetooth wireless arrangement or an IEEE 802.11 wireless arrangement, a Near Field Communication, NFC, arrangement or other suitable interface.

The I/O interfaces 208 and 213 may afford either or both of serial and parallel connectivity, the former typically being implemented according to the Universal Serial Bus (USB) standards and having corresponding USB connectors (not illustrated).

Storage devices 209 are provided and typically include a hard disk drive (HDD) 210. Other storage devices such as, an external HD 227, a disk drive (not shown) and a magnetic tape drive (not shown) may also be used. An optical disk drive 212 is typically provided to act as a non-volatile source of data. Portable memory devices, such as optical disks (e.g.: CD-ROM, DVD, Blu-Ray Disc), USB-RAM, external hard drives and floppy disks for example, may be used as appropriate sources of data to the debtor mobile computing device 200. Another source of data to debtor mobile computing device 200 is provided by the at least one server computer 292 through network 220.

The components 205 to 213 of the computer module 201 typically communicate via an interconnected bus 204 in a manner which results in a conventional mode of operation of debtor mobile computing device 200. In the embodiment shown in FIGS. 2A and 2B, processor 205 is coupled to system bus 204 through connections 218. Similarly, memory 206 and optical disk drive 212 are coupled to the system bus 204 by connections 219. Examples of a debtor mobile computing device 200 on which the described arrangements can be practiced include smart phones; tablet computers, gaming consoles, media players, TVs, wearable devices such as watches and glasses or a like device comprising a computer module like computer module 201. It is to be understood that when debtor mobile computing device 200 comprises a smart phone or a tablet computer some illustrated input and output devices may not be included such as, mouse pointer device 201; keyboard 202; scanner 226; and printer 215.

FIG. 2B is a detailed schematic block diagram of processor 205 and a memory 234. The memory 234 represents a logical aggregation of all the memory modules, including the storage device 209 and semiconductor memory 206, which can be accessed by the computer module 201 in FIG. 2A.

The methods of the invention may be implemented using debtor mobile computing device 200 wherein the methods may be implemented as one or more software application programs 233 executable within computer module 201. In particular, the steps of the methods of the invention may be effected by instructions 231 in the software carried out within the computer module 201.

The software instructions 231 may be formed as one or more code modules, each for performing one or more particular tasks. The software 233 may also be divided into two separate parts, in which a first part and the corresponding code modules performs the method of the invention, and a second part and the corresponding code modules manage a graphical user interface between the first part and the user.

The software 233 may be stored in a computer readable medium, including in a storage device of a type described herein. The software may load into the debtor mobile computing device 200 from the computer readable medium or through network 221 or 223, and then executed by debtor mobile computing device 200. In one example, the software 233 is stored on storage medium 225 that is read by optical disk drive 212. In another embodiment, the software 233 is stored in a server computer or cloud 291 and accessed by debtor mobile computing device 200. When software 233 is stored in debtor mobile computer device 200, typically it is stored in the HDD 210 or the memory 206.

A computer readable medium having such software 233 or computer program recorded on it is a computer program product. The use of the computer program product in the debtor mobile computing device 200 preferably effects a device or apparatus for implementing the methods of the invention.

In some instances, the software application programs 233 may be supplied to the user encoded on one or more disk storage medium 225 such as a CD-ROM, DVD or Blu-Ray disc, and read via the corresponding drive 212, or alternatively may be read by the user from the networks 220 or 222. Still further, the software can also be loaded into the debtor mobile computing device 200 from other computer readable media. Computer readable storage media refers to any non-transitory tangible storage medium that provides recorded instructions and/or data to the computer module 201 or debtor mobile computing device 200 for execution and/or processing. Examples of such storage media include floppy disks, magnetic tape, CD-ROM, DVD, Blu-ray Disc, a hard disk drive, a ROM or integrated circuit, USB memory, a magneto-optical disk, or a computer readable card such as a PCMCIA card and the like, whether or not such devices are internal or external of the computer module 201. Examples of transitory or non-tangible computer readable transmission media that may also participate in the provision of software application programs 233, instructions 231 and/or data to the computer module 201 include radio or infra-red transmission channels as well as a network connection 221, 223, 334, to another computer or networked device such as, server 291 and the Internet or an Intranet including email transmissions and information recorded on Websites and the like.

The second part of the application programs 233 and the corresponding code modules mentioned above may be executed to implement one or more graphical user interfaces (GUIs) to be rendered or otherwise represented upon display 214. Through manipulation of, typically, touchscreen 214 a user of debtor mobile computing device 200 and the methods of the invention may manipulate the interface in a functionally adaptable manner to provide controlling commands and/or input to the applications associated with the GUI(s). Other forms of functionally adaptable user interfaces may also be implemented, such as an audio interface utilizing speech prompts output via loudspeakers 217 and user voice commands input via microphone 280. The manipulations including screen touches, speech prompts, device movement gestures and/or user voice commands may be transmitted via network 220 or 222.

When the computer module 201 is initially powered up, a power-on self-test (POST) program 250 may execute. The POST program 250 is typically stored in a ROM 249 of the semiconductor memory 206. A hardware device such as the ROM 249 is sometimes referred to as firmware. The POST program 250 examines hardware within the computer module 201 to ensure proper functioning, and typically checks processor 205, memory 234 (209, 206), and a basic input-output systems software (BIOS) module 251, also typically stored in ROM 249, for correct operation. Once the POST program 250 has run successfully, BIOS 251 activates hard disk drive 210. Activation of hard disk drive 210 causes a bootstrap loader program 252 that is resident on hard disk drive 210 to execute via processor 205. This loads an operating system 253 into RAM memory 206 upon which operating system 253 commences operation. Operating system 253 is a system level application, executable by processor 205, to fulfil various high level functions, including processor management, memory management, device management, storage management, software application interface, and generic user interface.

Operating system 253 manages memory 234 (209, 206) in order to ensure that each process or application running on computer module 201 has sufficient memory in which to execute without colliding with memory allocated to another process. Furthermore, the different types of memory available in the debtor mobile computing device 200 must be used properly so that each process can run effectively. Accordingly, the aggregated memory 234 is not intended to illustrate how particular segments of memory are allocated, but rather to provide a general view of the memory accessible by computer module 201 and how such is used.

Processor 205 includes a number of functional modules including a control unit 239, an arithmetic logic unit (ALU) 240, and a local or internal memory 248, sometimes called a cache memory. The cache memory 248 typically includes a number of storage registers 244, 245, 246 in a register section storing data 247. One or more internal busses 241 functionally interconnect these functional modules. The processor 205 typically also has one or more interfaces 242 for communicating with external devices via the system bus 204, using a connection 218. The memory 234 is connected to the bus 204 by connection 219.

Application program 233 includes a sequence of instructions 231 that may include conditional branch and loop instructions. Program 233 may also comprise data 232 which is used in execution of the program 233. The instructions 231 and the data 232 are stored in memory locations 228, 229, 230 and 235, 236, 237, respectively. Depending upon the relative size of the instructions 231 and the memory locations 228-230, a particular instruction may be stored in a single memory location as depicted by the instruction shown in the memory location 230. Alternately, an instruction may be segmented into a number of parts each of which is stored in a separate memory location, as depicted by the instruction segments shown in the memory locations 228 and 229.

In general, processor 205 is given a set of instructions 243 which are executed therein. The processor 205 then waits for a subsequent input, to which processor 205 reacts by executing another set of instructions. Each input may be provided from one or more of a number of sources, including data generated by one or more of the input devices 202, 203, or 214 when comprising a touchscreen, data received from an external source across one of the networks 220, 222, data retrieved from one of the storage devices 206, 209 or data retrieved from a storage medium 225 inserted into the corresponding reader 212. The execution of a set of the instructions may in some cases result in output of data. Execution may also involve storing data or variables to the memory 234.

The disclosed arrangements use input variables 254 that are stored in the memory 234 in corresponding memory locations 255, 256, 257, 258. The described arrangements produce output variables 261 that are stored in the memory 234 in corresponding memory locations 262, 263, 264, 265. Intermediate variables 268 may be stored in memory locations 259, 260, 266 and 267.

The register section 244, 245, 246, the arithmetic logic unit (ALU) 240, and the control unit 239 of the processor 205 work together to perform sequences of micro-operations needed to perform “fetch, decode, and execute” cycles for every instruction in the instruction set making up the program 233. Each fetch, decode, and execute cycle comprises:
(a) a fetch operation, which fetches or reads an instruction 231 from memory location 228, 229, 230;
(b) a decode operation in which control unit 239 determines which instruction has been fetched; and
(c) an execute operation in which the control unit 239 and/or the ALU 240 execute the instruction.

Thereafter, a further fetch, decode, and execute cycle for the next instruction may be executed. Similarly, a store cycle may be performed by which the control unit 239 stores or writes a value to a memory location 232.

Each step or sub-process in the methods of the invention may be associated with one or more segments of the program 233, and may be performed by register section 244-246, the ALU 240, and the control unit 239 in the processor 205 working together to perform the fetch, decode, and execute cycles for every instruction in the instruction set for the noted segments of program 233.

One or more other debtor mobile computer device 290 may be connected to the communications network 220 as seen in FIG. 1A. Each such debtor mobile computer device 290 may have a similar configuration to debtor mobile computer device 200 comprising a computer module 201 and corresponding peripherals.

One or more other server computer 291 may be connected to the communications network 220. These server computers 291 respond to requests from debtor mobile computing device 200, 290 or other server computers (not shown) to provide information. Each server computer 291 may comprise or be associated with one or more database 292 (not shown).

The methods of the invention may alternatively be implemented in dedicated hardware such as one or more integrated circuits performing the functions or sub functions of the described methods. Such dedicated hardware may include graphic processors, digital signal processors, or one or more microprocessors and associated memories.

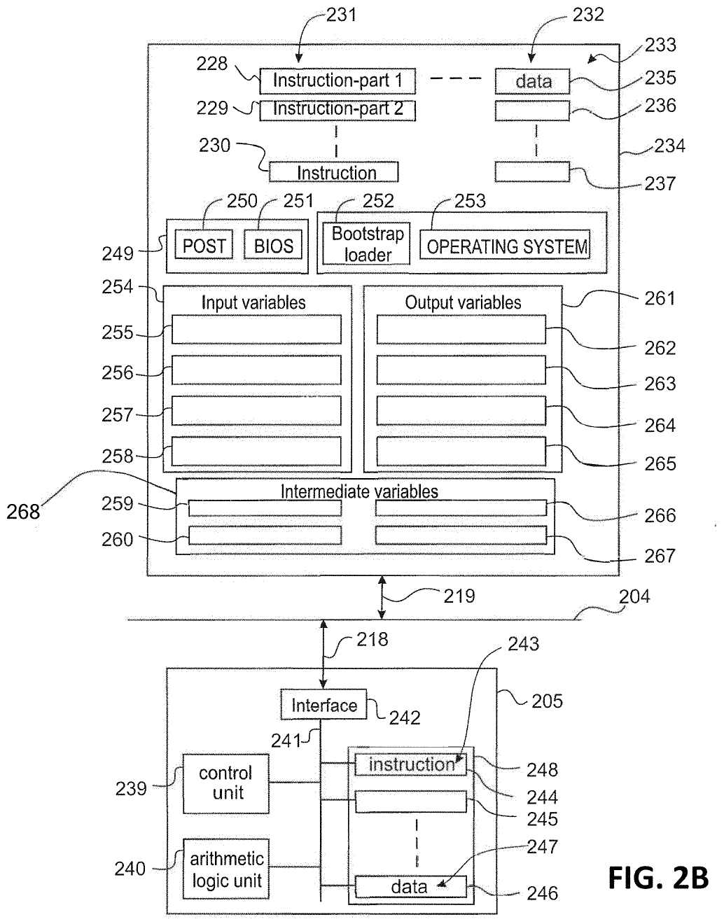

One embodiment of an operator dashboard 150 according to the invention is shown in FIG. 3. The operator dashboard 150 comprises menu bar 152 which is shown to comprise buttons such as, dashboard button 154, accounts button 156, settings button 156 and upload debts button 160. Also shown on dashboard 150 are recovered debts time frame display 162 and recovered debts total display 164.

Dashboard 150 further comprises a current status graphical display 166 and a current status numerical display 176. The current status graphical display 166 comprises recovered debts graphical display 168, payment plan graphical display 170, outstanding debts graphical display 172 and unrecoverable debts graphical display 174. The current status numerical display 176 comprises recovered debts numerical display 178, payment plan numerical display 180, outstanding debts numerical display 182 and unrecoverable debts numerical display 184.

The present invention is likely to find immediate application due to the high penetration of online banking in some markets and the large potential for application in markets where online penetration will increase, for example, the US and UK penetration rates are expected to rise from 60-70% towards 100%.

The present invention optimises the process of overdue account collection and the relationship between debtors and debtees.

Borrowers are frequently harassed over unintentional debts, many of which are simple oversights. The presently claimed invention addresses this by providing simple, straightforward ways for borrowers to keep track of what they owe. By focusing on a time saving, less confrontational, and easy to use self-management portal, borrower satisfaction may be increased and in turn the percentage of successful payments is increased. Another significant advantage of the present invention is that the automated method and system cannot be baited or biased when dealing with debtors.

So that the invention may be readily understood and put into practical effect, the following non-limiting example is provided.

### EXAMPLES

Next Best Action (NBA) Model

### Example Implementation in Toll Road Operator

### ‘Next Best Action ML Decisioning Model’ vs ‘Standard Linear Workflow’

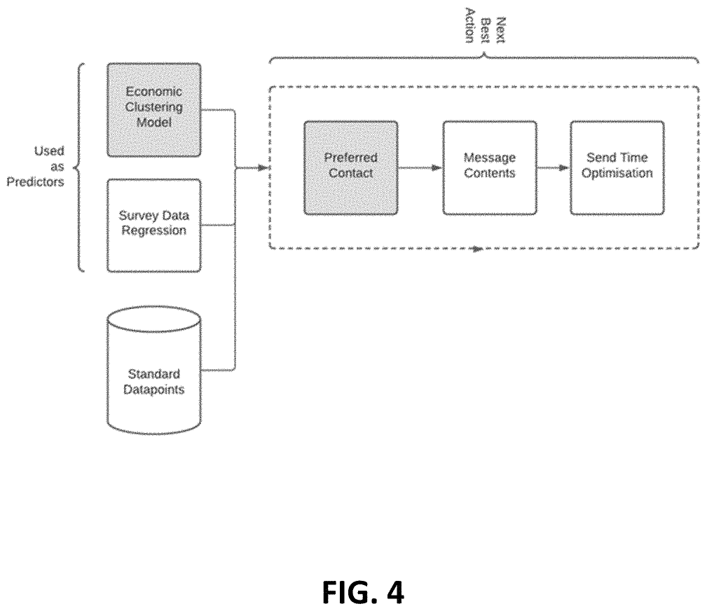

Mixture of SMS, email and dialler calls. All new referrals from initial date were split into champion and challenger segments. The NBA model outperformed the standard workflow by ˜35%, see Table 1 and FIGS. 5A and 5B.

TABLE 1
Toll Road Operator Results

| Campaign | Referrals | | | | | |
|---|---|---|---|---|---|---|
| Champion | 1164 | 78 | $156,862 | $3,371 | 6.70% | 2.15% |
| Challenger | 1157 | 105 | $151,333 | $5,505 | 9.08% | 3.64% |

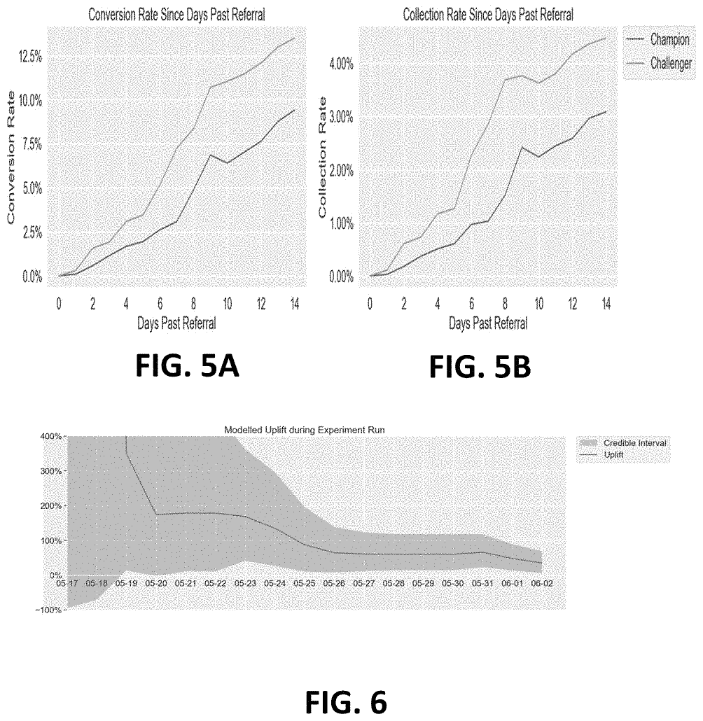

Modelled Uplift: based off results of an example implementation, the inventors NBA model outperforms the champion workflow with >=95% certainty. The finalised uplift is 35% with a 95% credible interval of between 5.7%-69.2%. The modelled credible interval over time is shown in FIG. 6.

### Example Implementation in Utility Service Provider

Overview and aim: This example implementation of the method of the invention demonstrated the effectiveness of digital collection strategies against non-digital or traditional phone call and mail collection strategies.

Design and implementation: the test was conducted utilising a batch of ‘low risk’ referrals from a Utility Service Provider. These referrals were then randomly assigned into one of three segments for the duration of the test:
Mixture of digital (and non-digital (letters and calls). This was the then current collections strategy;
Non digital (phone call and letters only);
Optimised digital (email and SMS);

All segments had similar debt distributions and customer profiles. All referrals used were at least 25 days past due on the week of the test starting. The duration of the test was 31 days.

Results: The most pertinent aspects of the results are summarised in the Table 2.

TABLE 1
Conversion and recovery rates by segment

| Segment | Digital (%) | Mixed (%) | Manual (%) |
|---|---|---|---|
| Conversion rate | 52.4 | 46.6 | 40.1 |
| (marginal) | | | |
| Recovery rate | 50.0 | 44.0 | 40.8 |
| (marginal) | | | |
| Mean recovery rate | 53.6 | 47.2 | 40.6 |
| (marginal) | | | |
| Mean recovery rate | 102.3 | 101.4 | 101.4 |
| (converted) | | | |

At the conclusion of the 31-day referral period, the digital-only strategy achieved a marginal conversion rate (fraction of referrals making any payment) of 52.4% compared to the current mixed strategy of 46.6% and the manual (non-digital only) strategy of 40.1%.

This was a 12.5% uplift in conversion for digital-only compared to the current mixed collection strategy, and a 30.8% uplift compared to the manual workflow. The differences in conversion rate for digital-only versus mixed and mixed versus manual workflows are both statistically significant.

The marginal recovery rate (total payments divided by total amount due) for digital was 50.0% compared to mixed at 44.0%, and manual at 40.8%. These marginal recovery rates have the same order as the conversion rates, but the differences are smaller because of a few referrals with very large amounts due obscuring the more general pattern.

The general pattern is better seen by looking at the marginal mean recovery rate, defined as the individual recovery rate (payments divided by amount due for each individual referral) averaged over all the referrals. The marginal mean recovery rate was 53.6% compared to mixed at 47.2%, and manual at 40.6%. These marginal mean recovery rates have the same order as the conversion rates, and the differences between segments are statistically significant.

However, mean recovery rate conditional on conversion, defined as the mean individual recovery rate calculated over only the referrals that converted, is very similar across all segments (slightly over 100%). None of the differences between segments is statistically significant. This means that for those referrals making any payment, there is no discernible difference between segments in the rate of recovery per referral. In other words, the differences between segments in marginal recovery rates are driven by the differences in conversion rates.

Conclusion: From the results of this experiment, collection strategies for this implementation utilizing digital collections are more likely to produce higher rates of conversion relative to non-digital strategies. The higher conversion rates from digital collection strategies advantageously lead to an increase in amount recovered due to the increase in the number of referrals making repayments.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

1. A method of determining a likelihood that a debt payment will be made or received, the method comprising:
sending a message to a debtor computing device comprising a link to a debtor portal;
displaying on the debtor computing device an indication of the debt owing;
displaying on the debtor computing device a proposed payment plan for settlement of the debt;
receiving an indication that the debtor accepts the proposed payment plan;
collecting and storing in one or more database, data and/or metadata on debtor activity;
collecting behavioural data drawn from existing accounting and loan management systems and using testing to determine a most effective message to send to a debtor, the testing utilising one or more algorithm which collect data on debtor behaviour, and a suggestive analytics framework which takes the data on debtor behaviour and determines a most effective communication for debt recovery, wherein the testing using one or more algorithm and the suggestive analytics framework finds a fewest number of contact points with a highest recovery rate;
using real-time analytics to analyse the debtor behaviour to identify a most efficient and effective communication with debtors;
learning from debtor behaviour, the learning comprising a Next Best Action model to predict one or more new behaviour based on the collected behavioural data to thereby determine the likelihood that a debt payment will be made or received.

2. The method of claim 1, wherein the message sent comprises an electronic message.

3. The method of claim 1 wherein the message further comprises an indication of the debt owing.

4. The method of claim 1 further comprising the step of offering a discount to the debtor if the discounted amount is paid within a limited time.

5. The method of claim 4, wherein the offer of a discount initiates an automated negotiation process with the debtor.

6. The method of claim 1 wherein the payment is received through the debtor portal.

7. The method of claim 1 further comprising using algorithmic learning to inform decisions on the type of credit offered to a debtor and/or to identify a debtor risk profile.

8. A server based system for receiving a debt payment comprising:
a network connected messaging processor for sending a message to a debtor computing device, the sent message comprising a link to a debtor portal;
a portal for displaying on the debtor computing device an indication of the debt owing and for displaying on the debtor computing device a proposed payment plan for settlement of the debt;
a server adapted to interface with the debtor computing device for receiving an indication that the debtor accepts the proposed payment plan;
a database for collecting and storing data and/or metadata on debtor activity;
the portal collecting behavioural data drawn from existing accounting and loan management systems and using testing to determine a most effective message to send to a debtor, the testing utilising one or more algorithm which collect data on debtor behaviour, and a suggestive analytics framework which takes the data on debtor behaviour and determines a most effective communication for debt recovery, wherein the testing using one or more algorithm and the suggestive analytics framework finds a fewest number of contact points with a highest recovery rate;
using real-time analytics to analyse debtor behaviour to identify a most efficient and effective communication with customers;
learning from debtor behaviour, the learning comprising a Next Best Action model to predict one or more new behaviour based on the collected behavioural data to thereby determine the likelihood that a debt payment will be made or received.

9. The server based system of claim 8, wherein the message sent by the processor comprises an electronic message.

10. The server based system of claim 8, wherein the message sent by the processor further comprises an indication of the debt owing.

11. The server based system according to claim 8, wherein the portal further offers a discount to the debtor if the discounted amount is paid within a limited time.

12. A computer program product for receiving a debt payment, the computer program product comprising a non-transitory computer usable medium and computer readable program code embodied on said non-transitory computer usable medium, the computer readable code comprising: computer readable program code devices (i) configured to cause the computer to send a message to a debtor computing device, the sent message comprising a link to a debtor portal;
computer readable program code devices (ii) configured to cause the computer to display on the debtor computing device an indication of the debt owing and to display on the debtor computing device a proposed payment plan for settlement of the debt; and
computer readable program code devices (iii) configured to cause the computer to receive an indication that the debtor accepts the proposed payment plan;
computer readable program code devices (iv) configured to cause the computer to collect and store in one or more database, data and/or metadata on debtor activity;
computer readable program code devices (v) configured to cause the computer to collect behavioural data drawn from existing accounting and loan management systems and use testing to determine a most effective message to send to a debtor, the testing utilising one or more algorithm which collect data on debtor behaviour and a suggestive analytics framework which takes the data on debtor behaviour and determines a most effective communication for debt recovery, wherein the testing using one or more algorithm and the suggestive analytics framework finds the fewest number of contact points with the highest recovery rate;
computer readable program code devices (vi) configured to learn from the debtor behaviour, the learning comprising a Next Best Action model to predict one or more new behaviour based on the collected behavioural data to thereby determine the likelihood that a debt payment will be made or received.

13. The computer program product of claim 12, wherein the message sent comprises an electronic message.

14. The computer program product of claim 12, wherein the message sent further comprises an indication of the debt owing.

15. The method of claim 1, wherein the testing comprises testing to determine the most effective ways to contact a debtor.

16. The method of claim 15, wherein the testing comprises multivariate testing.

17. The method of claim 16, wherein the testing comprises A/B testing or split testing.

18. The method of claim 2, wherein the electronic message comprises one or more of an SMS, an RCS or an Email message.

19. The method of claim 1 further comprising sending the communication identified to be the most efficient and effective to the debtor; and
receiving the debt payment.

20. The method of claim 1, wherein the NBA model is dynamically adjusted based specified data.

21. The method of claim 1, wherein the NBA model is dynamically adjusted based on events post loading.

22. The method of claim 1, wherein the NBA model is self correcting.

23. The method of claim 1, wherein message and communication is tailored towards the specific debtor and responses to events and/or interaction.

24. The method of claim 20, wherein the specified data comprises provided client information.

25. The method of claim 24, wherein the provided client information comprises one or more of: client type; debt type; one or more client attribute and debt delinquency.

26. The method of claim 1, wherein the NBA model may adjust one or more prediction based on provided debtor information such as, known demographic data; regional demographic data; and debt specifics.

27. The method of claim 1, wherein the NBA model may adjust one or more prediction based on one or more outcomes of past events such as, digital delivery success metrics; and dialler calls success metrics.

28. The method of claim 21, wherein the events post loading comprise debtor engagement.

29. The method of claim 1, wherein the NBA model may link payments to actions and measure how much a payment can be attributed to each action.

30. The method of claim 1, wherein the NBA model comprises a Recurrent Neural Network (RNN).
